(19) United States
(12) Patent Application Publication
(10) Pub. No.: US 2026/0011447 A1
(43) Pub. Date: Jan. 8, 2026

(54) NON-INVASIVE ASSESSMENT MODEL AND RISK ASSESSMENT METHOD FOR EXPOSURE RISK OF MANGANESE, SELENIUM, AND CALCIUM IN COMPLEX ENVIRONMENT

(71) Applicant: Wei Juan MA, BaoJi City (CN)

(72) Inventor: Wei Juan MA, BaoJi City (CN)

(21) Appl. No.: 18/939,190

(22) Filed: Nov. 6, 2024

(30) Foreign Application Priority Data
Jul. 5, 2024 (CN) 202410905043.8

(51) Int. Cl. G16H 50/30; G16H 15/00

(52) U.S. Cl. CPC G16H 50/30; G16H 15/00

(57) ABSTRACT

The present disclosure relates to the field of public health, and in particular to a non-invasive assessment model and method for exposure risk of manganese, selenium, and calcium in complex environment for human. Through non-invasive testing of contents of selenium, manganese, and calcium in hair, fingernails or toenails and simultaneous analysis of results of indicators MS, CS, and MC, risk categories and levels of multiple complex risks in human exposure to pollutant energy and environmental pollution, food and dietary supplement, medication and pharmaceutical preparations, new materials, occupational exposure, risk factors of endemic diseases, and the like are accurately assessed using mathematical function and probability, and risk indicators for surveillance are determined, so that the prevention strategy is implemented before clinical diagnosis, the incidence of diseases is decreased, and relevant standard is revised.

### CROSS-REFERENCE TO RELATED APPLICATIONS

This application claims priority to Chinese Patent Application No. 202410905043.8, filed on Jul. 5, 2024, which is hereby incorporated by reference its entirety.

### TECHNICAL FIELD

The present disclosure relates to public health and environmental exposure risk assessment.

### BACKGROUND

Manganese (Mn) is a potentially essential trace element with large variety sources from natural environment, and it was reported that manganese deficiency of human in natural causal are rare. Mseleni joint disease may be related to manganese deficiency, which has only been reported in northern Kwazulu Natal of South Africa, and the mseleni joint disease was once reported with short stature, short fingers and short toes. Long-term exposure to excessive manganese causes neurotoxicity, which mainly manifests as hyperirritability, violent acts and hallucinations, and then progressed to extrapyramidal symptoms similar to those of Parkinson disease, known as manganese-induced parkinsonism. Additionally, manganese may be involved in Alzheimer's disease.

Calcium (Ca) is a macroelement. Under natural conditions, calcium deficiency in human mainly manifests as impaired growth of skeletal system or component changes affect bone mass mineralization. Calcium excessive exposure may induced by other factors or when supplement excessively, but toxication is not reported yet.

Selenium (Se) is an essential trace element. For human, the range of selenium requirement from minimum to tolerable upper intake levels is narrow. When human has low selenium level may benefit from selenium supplementation. Oppositely, it may cause adverse effect when human has high selenium level.

Studies on the effects of selenium for human have demontrated that properly increased selenium level is beneficial to neurological and cardiovascular diseases. In China, selenium deficiency is considered as risk factor for Kashin-Beck disease and Keshan disease, and selenium was used in preventive measures and therapy. Selenosis caused by excessive selenium supplement have been reported in many countries. Symptoms of selenosis including: garlic odor; thickened, brittle nails with longitudinal streaks and white spots; dry, brittle hair and easily broken off at scalp; redness, swelling, blisters, and even ulcerate on the skin of hands and feet; severe dental caries and tooth decay; muscle or joint pain; and nervous system abnormalities mainly including numbness, paralysis, and convulsions, which vary in degree of severity. In recent years, it has been reported that chronic excessive selenium exposure may also cause amyotrophic lateral sclerosis.

Methods for testing manganese toxication and selenosis were originally established for the crowd who engaged in manganese and selenium mining and smelting. Indicators used to date to assess selenium status of human are testing the selenium concentration in nails and hair, additionally, testing blood and urinary selenium concentration in acute toxication. A short-term indicator for assessing manganese status of human is testing the manganese concentration in blood and urine. A short-term indicator for assessing a calcium status of human is testing the calcium concentration in blood and urine. There is no available long-term assessment indicators for manganese and calcium status in human.

For a long time, due to people in exposure are specific crowds, assessment of selenium and manganese has been confined to passive testing after exposure. With the development of economy and industry in human world, the risk of exposure factors caused by environmental pollution has expanded to all mankind, but early warning and initiative assessment method are not available in this field. In addition to directly affecting mining and smelting professionals, selenium and manganese minerals emerged on the earth surface can enter into surface water and into the surface water cycle eventually; they can enter into groundwater through infiltration, to pollute water sources in deep layer; they can enter into the soil and be utilized by plants, result in indirect high exposure in food sources; and mineral particles spread to other areas via air cycle. Main sources of selenium in environmental pollution are coal and petroleum. Currently, more than 50% of the world's energy supply comes from coal and petroleum. The mining and use of pollutant energy has caused exposure to risk factors since it was explored, but there is no comprehensive assessment system for human exposure risks worldwide.

### SUMMARY

To set up the first exposure risk assessment model and method in the world to assess manganese, selenium, and calcium exposure in ingestion toxication, endemic diseases, occupational exposure, and in the pollution caused by pollutant energy under the complex environment, technical solutions used in the present disclosure are as follows:
a non-invasive assessment model for exposure risk of manganese, selenium, and calcium in complex environment is established, using the following method:
a. sampled subject selection for the model: selecting sampled subjects from a healthy crowd, a crowd in selenium deficiency environment, a crowd in selenium toxicity environment, a crowd in manganese deficiency environment, a crowd in manganese toxicity environment, and a crowd in calcium deficiency environment.
b. data collection: collecting manganese content data, selenium content data, and calcium content data of each sampled subject from hair, toenails or fingernails of the sampled subject;
c. data processing: dividing the collected manganese content data, selenium content data, and calcium content data of each sampling subject in elements pairs to obtain modeling data, or before obtaining the modeling data, scaling at least one element selected from the manganese content data, selenium content data, and calcium content data and then dividing the scaled manganese content data, selenium content data, and calcium content data in elements pairs to obtain the modeling data; wherein processing in division between any two elements of the manganese content data, selenium content data, and calcium content data in the modeling data of different sampled subjects are consistent; and
d. establishing an exposure risk assessment model: using modeling data of sampled subjects from different crowds to establish a group of discriminant equation, to obtain the exposure risk assessment model.

Further, during data processing, modeling data is obtained using ratios of manganese/selenium, manganese/calcium, and calcium/selenium.

Further, during data processing, calcium content data collected from each sampled subject is scaled down.

Further, the calcium content data collected from each sampled subject is scaled down by 5 to 10,000 times.

Further, the calcium content data collected from each sampled subject is scaled down by 100 times.

Further, during data processing, the manganese content data and the selenium content data collected from each sampled subject are scaled up.

Further, the manganese content and the selenium content collected from each sampled subject are scaled up by 5 to 10,000 times.

Further, the manganese content and the selenium content collected from each sampled subject are scaled up by 100 times.

Further, a linear regression equation is established using the modeling data from exposure risk assessment model, and a risk level assessment model is obtained.

According to the above established model, the exposure risk is assessed, including the following method:
a. collecting manganese content data, selenium content data, and calcium content data from hair, toenails, or fingernails of an subject in assessing;
b. collecting the manganese content data, selenium content data, and calcium content data of the subject in assessing consist with the process of modeling data (the manganese content data, selenium content data, and calcium content data) when the exposure risk assessment model is established, to obtain assessment data; and
c. substituting the assessment data into the group of discriminant equation for calculation, and determining a primary risk category.

Further, a result of the group of discriminant equation is used to determine the primary risk category through a posterior probability.

Further, a linear regression equation is established using the modeling data from exposure risk assessment model, and a risk level assessment model is obtained, and the assessment data is substituted into the linear regression equation for calculation to determine the level of the primary risk.

Further, abnormal result of indicator is determined according to confidence interval (a reference value range) of single element and confidence interval of a ratio between elements in pairs, to determine a secondary risk.

Further, the manganese content data, selenium content data, and calcium content data are collected from the subject in assessing through instrument, and the collected manganese content data, selenium content data, and calcium content data are calculated and processed by a computer program and then substituted into the exposure risk assessment model in the program to output an assessment report.

Further, the manganese content data, selenium content data, and calcium content data collected by the instrument is manually input into the computer program or directly transmitted to the computer program.

Further, the assessment report is output via a printer or displayed on a display device.

Further, the instrument for collecting the manganese content data, selenium content data, and calcium content data is a mass spectrometer or a spectrometer.

Advantages and beneficial effects of the present disclosure including:

(1) When there are multiple exposure risks in the environment, the major risk factor can be determined in the complex exposure situation and different with the minor one; and long-term exposure of human can be assessed.

(2) The assessment method has high sensitivity and can assess the environmental exposure risk before disease diagnosis.

(3) The assessment system is comprehensive and can assess six type exposure risks of human simultaneously under one testing event.

(4) The tested specimen are collected non-invasively and the collection manner is easy to be accepted; the specimen are stable and a transportation process is not affected by time and environmental factors.

(5) Exposure risk assessment of the crowd for ingestion helps monitor and assess the results after ingestion, thereby standardize the food and dietary supplements, standardize manufacture, pharmaceutical preparations, materials and medication treatment.

(6) Exposure risk assessment of the crowd affected by occupational and local factors helps reduce occupational exposure disease and endemic diseases.

(7) Exposure risk assessment of the crowd in environmentally polluted areas helps to supervise the management of pollutant energy, thereby reducing the mining and using of pollutant energy, and stop poor quality coal and petroleum entering the market; and also helps promote the development and application of green energy and pollution-free energy.

### BRIEF DESCRIPTION OF THE DRAWINGS

FIG. 1 is a schematic diagram of distribution patterns of environmental exposure risks of MS, SC, and MC. Where k=1, it is determined to be a healthy person; k=2, it is determined to be an exposure risk of selenium deficiency in environment; k=3, it is determined to be an exposure risk of selenium toxicity in environment; k=4, it is determined to be an exposure risk of manganese deficiency in environment; k=5, it is determined to be an exposure risk of manganese toxicity in environment; and k=6, it is determined to be an exposure risk of calcium deficiency in environment. In the case of a single exposure risk of Mn, Se, or Ca, a primary category data set distributed in an assessment system is a primary risk. In the complex case of multiple exposure risks, actual data obtained is distributed in the primary category data set (primary risk indicator) or combined with trend toward secondary risk indicator. As the level of the exposure risk is increased, an assessed result deviates from healthy person pattern more severely and migrates toward the risk.

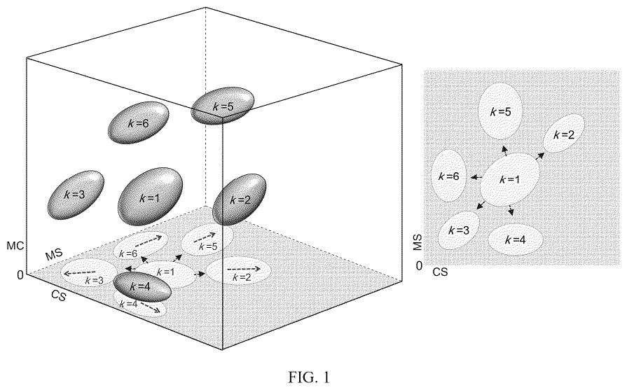

### DESCRIPTION OF THE EMBODIMENTS

It is found through study that there are balance relationships within manganese, selenium, and calcium, especially the linear relationship expressed in exposure risks. In the case of a single environmental exposure risk, a change in the single exposure factor expresses as an increase or decrease. However, the actual situation of environmental risk exposure is usually complex, and variety risk exposure factors may be co-effect according to contact factor, living environment, and working environment. In the case of the multiple risk exposure factors, a previous single factor of Mn, Se, or Ca can determine only an certain exposure risk of one factor, but cannot conclude which risk factor is major one. Assessment using new indicators MS, CS, and MC can determine the major risk factor and different with minor one in complex exposure situation. In addition, these three new indicators have higher sensitivity and higher accuracy in assessing exposure risks than testing manganese, selenium, and, calcium separately. The new indicators and single factors are used to non-invasively test hair, fingernails, and toenails, and according to the results of this test, an assessment system for human long-term exposure risks is created, which is the first exposure risk assessment system worldwide to assess the exposure risk in ingestion toxication, endemic diseases, occupational exposure, and environmental pollution caused by pollutant energy.

An assessment model and assessment method are introduced in detail below.

Assessment of human exposure risks of manganese, selenium, and calcium in complex environment using non-invasive indicators MS, CS, MC and single factor testing, which includes six categories. Because children mostly are affected by environmental exposure and growth factors rather than occupational exposure, exposure risk assessment systems of children and adults are established separately according to differences in exposure conditions.

1. Assessment system indicators:

Exposure risk assessment and testing includes non-invasively collecting hair, fingernails, and toenails from sampled subjects, and analyzing separately by specimen types.

### (1) Single Factor Indicator

The contents of manganese (Mn), selenium (Se), and calcium (Ca) of specimen are analyzed in the same time and recorded as Mn (μg/g), Se (μg/g), and Ca (μg/g) respectively.

### (2) New Indicators

MS: A test value of manganese (Mn) in a specimen is recorded as M, a test value of selenium (Se) in the same specimen is recorded as S, and a ratio of M to S is recorded as MS (M/S).

$$MS = \frac{Mn ( µg / g )}{Se ( µg / g )}$$

CS: A test value of calcium (Ca) in a specimen is recorded as C, one percent of Cis recorded as Ca′, a test value of selenium (Se) in the same specimen is recorded as S, and a ratio of C to S is recorded as CS (C/S).

$${Ca}^{′} = \frac{Ca ( µg / g )}{100} \times 100$$

$$CS = \frac{Ca ( µg / g )}{Se ( µg / g )} = \frac{{Ca}^{′} ( µg / g )}{Se ( µg / g )} = \frac{Ca ( µg / g ) / 100}{Se ( µg / g )} \times 100$$

MC: A test value of manganese (Mn) in a specimen is recorded as M, a test value of calcium (Ca) in the same specimen is recorded as C, one percent of C is recorded as Ca′, and a ratio of M to C is recorded as MC (M/C).

$${Ca}^{′} = \frac{Ca ( µg / g )}{100} \times 100$$

$$MC = \frac{Mn ( µg / g )}{Ca ( µg / g )} = \frac{Mn ( µg / g )}{{Ca}^{′} ( µg / g )} = \frac{Mn ( µg / g )}{Ca ( µg / g ) / 100} / 100$$

Note: for all test values, when assessment and calculation are performed, the new indicators may be in the form of any ratio in pairs of manganese content data, selenium content data, and calcium content data, or a mathematical form of the ratio form is equivalent to the new indicators in meaning, such as selenium/manganese, selenium/calcium, and calcium/manganese. In specific implementation, the above forms of manganese/selenium, calcium/selenium, and manganese/calcium are optimal.

Because manganese and selenium are trace elements in human, but calcium is a macroelement, the values are not convenient to be recorded when manganese and selenium are respectively divided by calcium. To facilitate analyst to read the new indicator values, a better way is to scale at least one element selected from the collected manganese content data, selenium content data, and calcium content data and then perform a division of the scaled content data in pairs. The scaling means scaling down or scaling up sampling values of one or several elements. The calcium content data may be usually scaled down, such as taking a value of one-fifth to one ten-thousandth of the calcium content data as a mathematical form of the defined indicator, and taking a value of one percent of the calcium content data for calculation is optimal, to facilitate numerical reading. Alternatively, the manganese content data and selenium content data are scaled up, such as scaling up the manganese content data and selenium content data by 5 to 10,000 times, and scaling up the manganese content data and selenium content data by 100 times is optimal. Of course, using the above scaling of the manganese content data, selenium content data, and calcium content data and then dividing the scaled content data in pairs, and using original data in dividing the manganese content data, selenium content data, and calcium content data in pairs have same assessment result in a model assessment and should be regarded as equivalent. The following description is according to the optimal forms of the above indicators.

2. Main categories of the assessment system:

### (1) Healthy

### (2) Exposure Risk of Selenium Deficiency in Environment:

Selenium deficiency in environment result in insufficient intake by human (selenium deficiency in food, drinking water, geographical environment, or the like).

### (3) Exposure Risk of Selenium Toxicity in Environment:

Selenium toxicity in environment result in excessive intake exposure (excessive food and dietary supplement with selenium; use of selenium-containing preparations, selenium-containing medication, and selenium-containing materials), excessive occupational exposure (selenium mining, smelting, processing and transportation; coal mining and use; petroleum mining, crude smelting and use; manufacturing of selenium-containing preparations, selenium-containing pharmaceuticals, and selenium-containing materials), and environmental pollution exposure risks (discharge or emissions during mining, smelting, and processing).

### (4) Exposure Risk of Manganese Deficiency in Environment:

Manganese deficiency in environment result in insufficient intake by human (manganese deficiency in food, drinking water, geographical environment, or the like).

### (5) Exposure Risk of Manganese Toxicity in Environment:

Manganese toxicity in environment result in excessive intake exposure (excessive food and dietary supplement with manganese, use of manganese-containing preparations, manganese-containing medication, and manganese-containing materials), excessive occupational exposure (manganese mining, smelting, processing and transportation; manganese industry; manufacturing of manganese-containing preparations, manganese-containing pharmaceuticals, and manganese-containing materials), and environmental pollution exposure risks (discharge or emissions during mining, smelting, and processing).

### (6) Exposure Risk of Calcium Deficiency in Environment:

Calcium deficiency in environment result in insufficient intake and storage of calcium in human (from food, drinking water, and the like).

3. Classification of primary exposure risks of assessed subjects, which is determined by mathematic function and probability.

### (1) Establishing an Exposure Risk Assessment Model:

Model conditions for an exposure risk assessment system with new indicators MS, CS, and MC:
Sampled object crowds are selected according to the above six risk categories:
Healthy:

When selecting a healthy crowd to collect data for assessment system model need to meet the following requirements: \textcircled{1}. good health, no suffering disease; \textcircled{2}. no history of medication treatment in the past one month; \textcircled{3}. no nutritional dietary supplements related to manganese, selenium and calcium, no functional foods containing with manganese, selenium and calcium, and no specific materials composed of manganese, selenium and calcium have been used in the past one month; \textcircled{4}. being engaged in an occupation not related to exposure risks; \textcircled{5}. not living in endemic disease area in the past half year; and \textcircled{6}. not living in an area around environmental exposure risks in the past three months.

### Exposure Risk of Selenium Deficiency in Environment:

When selecting a crowd who exposure in selenium deficiency environment to collect data for assessment system model need to meet the following items: \textcircled{1}. (required item) primary selenium deficiency with clinical diagnosis, or combined with any one from \textcircled{2}, \textcircled{3}, \textcircled{4} and \textcircled{5}; \textcircled{2}. suffering from a selenium deficiency-related disease; \textcircled{3}. having a history of a selenium deficiency-related disease; \textcircled{4}. living in an endemic selenium deficiency diseases area in the past half year; \textcircled{5}. family members living together are suffering from or have a history of a selenium deficiency-related disease.

### Exposure Risk of Selenium Toxicity in Environment:

When selecting a crowd who exposure in selenium toxicity environment to collect data for assessment system model need to meet the following items: \textcircled{1}). (required item) primary selenosis with clinical diagnosis, or combined with any one from \textcircled{2}, \textcircled{3}, \textcircled{4} and \textcircled{5}; \textcircled{2}. having a history of exposure to selenium supplement, nutritional preparations, medication treatments, or materials containing selenium within the past one month; \textcircled{3}. occupational exposure: selenium mining, smelting, processing and transportation; coal mining and use; petroleum mining, crude smelting and use; and manufacturing of preparations, pharmaceuticals, and materials containing selenium; \textcircled{4}. living in surrounding areas of selenium mining and energy mining, processing, use and energy production industries within the past one month; and \textcircled{5}. family members living together have occupational exposure to selenium.

### Exposure Risk of Manganese Deficiency in Environment:

When selecting a crowd who exposure in manganese deficiency environment to collect data for assessment system model need to meet the following items: \textcircled{1}. (required item) primary manganese deficiency with clinical diagnosis, or combined with any one from \textcircled{2}, \textcircled{3}, \textcircled{4} and \textcircled{5}; \textcircled{2}. suffering from manganese deficiency-related diseases; \textcircled{3}. having a history of manganese deficiency-related diseases; \textcircled{4}. living in an endemic manganese deficiency diseases area in the past half year; and \textcircled{5}. family members living together are suffering from or have a history of manganese deficiency-related diseases.

### Exposure Risk of Manganese Toxicity in Environment:

When selecting a crowd who exposure in manganese toxicity environment to collect data for assessment system model need to meet the following items: \textcircled{1}. (required item) primary manganese toxication with clinical diagnosis, or combined with any one from \textcircled{2}, \textcircled{3}, \textcircled{4} and \textcircled{5}; \textcircled{2}. having a history of exposure to manganese supplement, nutritional preparations, medication treatments, and specific materials containing manganese within the past one month; \textcircled{3}. occupational exposure: manganese mining, smelting, processing and transportation; and industrial manufacturing, preparations, pharmaceuticals, and materials containing manganese; \textcircled{4}. living in surrounding areas of manganese mining, smelting, processing, and use within the past one month; and \textcircled{5}. family members living together are occupationally exposed to manganese.

### Exposure Risk of Calcium Deficiency in Environment:

When selecting a crowd who exposure in calcium deficiency environment to collect data for assessment system model need to meet the following items: \textcircled{1}. (required item) primary calcium deficiency with clinical diagnosis, or combined with any one from \textcircled{2}, \textcircled{3}, and \textcircled{4}; \textcircled{2}. suffering from calcium deficiency-related diseases; \textcircled{3}. having a history of calcium deficiency-related diseases; and \textcircled{4}. family members living together are suffering from or have a history of calcium deficiency-related diseases.

The manganese content data, selenium content data and calcium content data are collected from hair, toenails, or fingernails of the sampled subjects from the above different crowds, and modeling data is obtained after the data is processed according to the above new indicators MS, CS, and MC.

The new indicators MS, CS, or MC are Xⱼ (j=1, 2, and 3. To be specific, X₁ is modeling data when the new indicator is MS, X₂ is modeling data when the new indicator is CS, and X₃ is modeling data when the new indicator is MC); A main category is k, (k=1, 2, 3, 4, 5, and 6, To be specific, k=1, healthy; k=2, an exposure risk of selenium deficiency in environment; k=3, an exposure risk of selenium toxicity in environment; k=4, an exposure risk of manganese deficiency in environment; k=5, an exposure risk of manganese toxicity in environment; and k=6, an exposure risk of calcium deficiency in environment. Indicator data is Xᵢ (i=1, 2, 3, 4, 5, . . . , and n, that is, the number of modeling data of sampled subjects in different crowds, and each group do not be required has same cases number.):

A pooled covariance Sᵢⱼ of Xᵢ and Xⱼ is calculated and listed in a matrix S:

$${S}_{ij} = \frac{{\sum }_{k = 1}^{G} {\sum }_{t = 1}^{{n}_{k}} ( {X}_{it}^{k} - {\overline{X}}_{i}^{k} ) ( {X}_{jt}^{k} - {\overline{X}}_{j}^{k} )}{{\sum }_{k = 1}^{G} ( {n}_{k} - 1 )}$$

Where X̅ᵢ^{k} and X̅ⱼ^{k} are mean numbers of variables Xᵢ and Xⱼ in a k category, and nₖ is the number of cases in the k category.

$$S = [ \begin{matrix}{S}_{11} & {S}_{12} & {S}_{13} \\ {S}_{21} & {S}_{22} & {S}_{23} \\ {S}_{31} & {S}_{32} & {S}_{33}\end{matrix} ]$$

A discrimination coefficient Cⱼₖ (j=0, 1, 2, and 3; k=1, 2, 3, 4, 5, and 6) is calculated using the matrix S.

$$\{ \begin{matrix}{S}_{11} {C}_{1 k} + {S}_{12} {C}_{2 k} + {S}_{13} {C}_{3 k} = {{\overline{X}}_{1}}^{( k )} \\ {S}_{21} {C}_{1 k} + {S}_{22} {C}_{2 k} + {S}_{23} {C}_{3 k} = {{\overline{X}}_{2}}^{( k )} \\ {S}_{21} {C}_{1 k} + {S}_{32} {C}_{2 k} + {S}_{33} {C}_{3 k} = {{\overline{X}}_{3}}^{( k )}\end{matrix}$$

According to the six categories, six sets of equations are generated for calculation, generating C₁ₖ, C₂ₖ, and C₃ₖ for each group, namely:
when k=1, C₁₁, C₂₁, and C₃₁ are obtained according to the equations;
when k=2, C₁₂, C₂₂, and C₃₂ are obtained according to the equations;
when k=3, C₁₃, C₂₃, and C₃₃ are obtained according to the equations.
when k=4, C₁₄, C₂₄, and C₃₄ are obtained according to the equations;
when k=5, C₁₅, C₂₅, and C₃₅ are obtained according to the equations;
when k=6, C₁₆, C₂₆, and C₃₆ are obtained according to the equations.

A prior probability P(Yₖ) and a discrimination coefficient C₀ₖ are calculated as follows:

$$P ( {Y}_{k} ) = \frac{{n}_{k}}{N}$$

$${C}_{0 k} = log P ( {Y}_{k} ) - \frac{1}{2} {\sum }_{j = 1}^{m} {C}_{jk} {\overline{X}}_{j}^{( k )}$$

And C₀₁, C₀₂, C₀₃, C₀₄, C₀₅, and C₀₆ are obtained through calculation.

A group of discriminant equation is established, that is, the exposure risk assessment model is obtained:

$$\{ \begin{matrix}{Y}_{1} = {C}_{01} + {C}_{11} {X}_{1} + {C}_{21} {X}_{2} + {C}_{31} {X}_{3} \\ {Y}_{2} = {C}_{02} + {C}_{12} {X}_{1} + {C}_{22} {X}_{2} + {C}_{32} {X}_{3} \\ {Y}_{3} = {C}_{03} + {C}_{13} {X}_{1} + {C}_{23} {X}_{2} + {C}_{33} {X}_{3} \\ {Y}_{4} = {C}_{04} + {C}_{14} {X}_{1} + {C}_{24} {X}_{2} + {C}_{34} {X}_{3} \\ {Y}_{5} = {C}_{05} + {C}_{15} {X}_{1} + {C}_{25} {X}_{2} + {C}_{35} {X}_{3} \\ {Y}_{6} = {C}_{06} + {C}_{16} {X}_{1} + {C}_{26} {X}_{2} + {C}_{36} {X}_{3}\end{matrix}$$

Note: the group of discriminant equation provided in this embodiment is Bayes discriminant, which is easy to implement. However, when the exposure risk assessment model is actually established, there is no limitation to the discriminant function form. The exposure risk assessment model may be obtained by using the Fisher discriminant for calculation. Because the calculation of the Fisher discriminant is relatively complicated, a specific calculation process is not repeated herein.

According to the above exposure risk assessment model, risk assessment can be performed on the subjects in assessing:

### (2) Assessing a Primary Risk Category of Unknown Subjects:

a scope of the subjects in assessing is random and non-selective.

Manganese content data, selenium content data, and calcium content data are obtained from hair, toenails, and fingernails of the subjects in assessing. After the above data is processed, values of the new indicators MS, CS, and MC are obtained, and categories are determined by the discriminant equation:

a. the values are substituted into the group of discriminant equation to calculate a function value Yₖ, and the subject is classified into a category with the largest mathematical function value.

b. a posterior probability Pₖ of the k category: the subject is classified into a category with the largest posterior probability (g=6).

$${Y}_{C} = Max ( {Y}_{k} )$$

$${P}_{k} = \frac{exp ( {Y}_{k} - {Y}_{c} )}{{Σ}_{l = 1}^{ℊ} exp ( {Y}_{l} = {Y}_{c} )}$$

k=1, which means that the unknown assessed subject is a healthy person, and auxiliary information reference is: having all model conditions \textcircled{1}, \textcircled{2}, \textcircled{3}, \textcircled{4}, \textcircled{5}, and \textcircled{6} of the healthy (the auxiliary information reference conditions are the same as the items listed in the above sampled subject crowd, and the following is similar).

k=2, which means that the unknown assessed subject is in a category of exposure risk of selenium deficiency in environment, and auxiliary information reference is: having any one item from \textcircled{1}, \textcircled{2}, \textcircled{3}, \textcircled{4}, and \textcircled{5} in exposure risk model of selenium deficiency in environment.

k=3, which means that the unknown assessed subject is in a category of exposure risk of selenium toxicity in environment, and auxiliary information reference is: having any one item from \textcircled{1}, \textcircled{2}, \textcircled{3}, \textcircled{4}, and \textcircled{5} in exposure risk model of selenium toxicity in environment.

k=4, which means that the unknown assessed subject is in a category of exposure risk of manganese deficiency in environment, and auxiliary information reference is: having any one item from \textcircled{1}, \textcircled{2}, \textcircled{3}, \textcircled{4}, and \textcircled{5} in exposure risk model of manganese deficiency in environment.

k=5, which means that the unknown assessed subject is in a category of exposure risk of manganese toxicity in environment, and auxiliary information reference is: having any one item from \textcircled{1}, \textcircled{2}, \textcircled{3}, \textcircled{4}, and \textcircled{5} in exposure risk model of manganese toxicity in environment.

k=6, which means that the unknown assessed subject is in a category of exposure risk of calcium deficiency in environment, and auxiliary information reference is: having any one item from \textcircled{1}, \textcircled{2}, \textcircled{3}, and \textcircled{4} in exposure risk model of calcium deficiency in environment.

4. The level of the primary exposure risk of the subject is assessed using a mathematical function.

The mathematical function value is used to assess the level which the subject is near to disease status after being exposed to a risk factor, which able to get measurable progression of exposure in assessment. A risk level assessment model uses assessment category determination data and model conditions.

### (1) Establishing the Risk Level Assessment Model:

the modeling data of the exposure risk assessment model are used in level assessment. New indicators MS, CS, and MC are Xⱼₘ(j, m=1, 2, and 3); the primary category is k (k=1, 2, 3, 4, 5, and 6); the indicator data is Xᵢ (i=1, 2, 3, 4, 5, . . . , and n); when k=6,

$${X}_{3} = \frac{1}{{X}_{i}}$$

is determined; the risk critical value of dependent variable of healthy group is Yₐ, and the risk critical value of dependent variable of an exposed group is Y_{b}.

A sum of squares of deviations from mean and a sum of products of deviations from mean are calculated respectively:

$${l}_{jm} = {\sum }_{i = 1}^{n} ( {X}_{ij} - {\overline{X}}_{j} ) ( {X}_{im} - {\overline{X}}_{m} ) = {\sum }_{i = 1}^{n} {X}_{ij} {X}_{im} - \frac{( {Σ}_{i = 1}^{n} {X}_{ij} ) ( {Σ}_{i = 1}^{n} {X}_{im} )}{n}$$

$${l}_{jy} = {\sum }_{i = 1}^{n} ( {X}_{ij} - {\overline{X}}_{j} ) ( {Y}_{i} - \overline{Y} ) = {\sum }_{i = 1}^{n} {X}_{ij} {Y}_{i} - \frac{( {Σ}_{i = 1}^{n} {X}_{ij} ) ( {Σ}_{i = 1}^{n} {Y}_{i} )}{n}$$

a deviation matrix L is established as follows:

$$L = [ \begin{matrix}{l}_{11} & {l}_{12} & {l}_{13} & {l}_{1 y} \\ {l}_{21} & {l}_{22} & {l}_{23} & {l}_{2 y} \\ {l}_{31} & {l}_{32} & {l}_{33} & {l}_{3 y} \\ {l}_{y 1} & {l}_{y 2} & {l}_{y 3} & {l}_{y y}\end{matrix} ]$$

An equation set is established according to the deviation matrix:

$$\{ \begin{matrix}{l}_{11} {β}_{1} + {l}_{12} {β}_{2} + {l}_{13} {β}_{3} = {l}_{1 y} \\ {l}_{21} {β}_{1} + {l}_{22} {β}_{2} + {l}_{23} {β}_{3} = {l}_{2 y} \\ {l}_{31} {β}_{1} + {l}_{32} {β}_{2} + {l}_{33} {β}_{3} = {l}_{3 y}\end{matrix}$$

Regression coefficients β₁, β₂, and β₃ are obtained according to the equation set.

A constant term β₀ is calculated.

$${β}_{0} = \overline{Y} - ( {β}_{1} {\overline{X}}_{1} + {β}_{2} {\overline{X}}_{2} + {β}_{3} {\overline{X}}_{3} )$$

A regression model is established:

$$Y = {β}_{0} + {β}_{1} {X}_{1} + {β}_{2} {X}_{2} + {β}_{3} {X}_{3}$$

### (2) Assessing the Primary Risk Level of the Unknown Subjects in Assessing:

new indicator results of the unknown subject in assessing are substituted into the established regression equation to calculate Yₓ:

\textcircled{1}. exposure risk Yₐ≤Yₓ≤Y_{b}, and when

$${Y}_{x} \geq {Y}_{\frac{a + b}{2}} ,$$

its exposure risk increase, and as Yₓ is larger, the exposure risk is greater, and with more possibility of clinical symptom occurrence.

Where, the upper limit of Yₓ is Y_{xU}, which is the actual upper limit of confirmed case in clinical diagnosis.

The lower limit of Yₓ is Y_{xL}, which is the actual lower limit of the healthy crowd.

\textcircled{2}. exposure risk Y_{b}≤Yₓ≤Yₐ, and when

$${Y}_{x} \leq {Y}_{\frac{a + b}{2}} ,$$

its exposure risk increase, and as Yₓ is smaller, the exposure risk is greater, and with more possibility of clinical symptom occurrence.

Where, the upper limit of Yₓ is Y_{xU}, which is the actual upper limit of the healthy crowd.

The lower limit of Yₓ is Y_{xL}, which is the actual lower limit of confirmed case in clinical diagnosis.

5. Abnormal results in indicators those needed to be surveillanced for the assessed subjects: primary risk indicators, secondary risk indicators, and reference indicators.

### (1) Establishing Confidence Interval of the Indicators:

the new indicators MS, CS, and MC and indicators Se, Mn, and Ca are Xⱼ (j=1, 2, 3, 4, 5, and 6); indicator data is Xᵢ; the number of cases is n (i=1, 2, 3, 4, 5, . . . , and n); an upper limit Uₖⱼ and a lower limit Lₖⱼ (k=1) of confidence interval of MS, CS, MC, S, M, and C of the healthy crowd are calculated respectively:

$${S}_{j} = \sqrt{\frac{{Σ ( {X}_{ij} - {\overline{X}}_{j} )}^{2}}{n - 1}} = \sqrt{\frac{{Σ ( {X}_{ij} )}^{2} - \frac{{( {ΣX}_{ij} )}^{2}}{n}}{n - 1}}$$

$$\begin{matrix}{U}_{kj} = {\overline{X}}_{j} + {{u}_{α}}_{/ 2} {S}_{j} \\ {L}_{kj} = {\overline{X}}_{j} - {{u}_{α}}_{/ 2} {S}_{j} \\ {L}_{kj} = {\overline{X}}_{j} - {u}_{α} {S}_{j}\end{matrix}$$

\textcircled{1}. when the environmental exposure risk category includes exposure deficiency and exposure toxicity, ua/2 is used for calculation, a 95% confidence interval is used as a reference, and ua/2=1.96 is determined; or a 99% confidence interval is used as a reference, and ua/2=2.58 is determined;

A confidence interval of the healthy crowd is (L₁ⱼ, U₁ⱼ).

\textcircled{2}. when the environmental exposure risk category includes only the exposure deficiency, uₐ is used for calculation, a 95% confidence interval is used as a reference, and uₐ=1.64 is determined; or a 99% confidence interval is used as a reference, and uₐ=2.33 is determined;

A confidence interval of the healthy crowd is (L₁ⱼ, Uₘₐₓ).

### (2) Abnormal Results in Indicators of the Unknown Assessed Subjects:

For the unknown assessed subjects, assessment is performed by comparing an actually obtained value Xⱼ with a corresponding (L₁ⱼ, U₁ⱼ) or (L₁ⱼ, Uₘₐₓ).

### 6. Comprehensively Assessing the Environmental Exposure Risk for the Unknown Subjects

Indicators MS, CS, and MC are used, mathematical function and probability are used for assessment and calculation, which can preferentially determine the category and level of the most severe human exposure risk in compex exposure risk situation. The assessment system preferentially determines: the primary risk indicator is the one which has abnormal result in a category; the secondary risk indicator is the one which has abnormal result beyond the category; and reference indicator is the single factor exposure beyond the category, thereby implementing comprehensive assessment of the environmental exposure risk.

In the case of different environmental exposure risks, each indicator is changed as shown in Table 1 when exposed to a single risk (− means normal; ↑ means increase; ↓ means decrease; and x results are unknown), and x represents a combined secondary risk index when multi-risk exposures occur and the reference index.)

TABLE 1
Changes of indicators in single environmental
exposure risk of Mn, Se, and Ca

| Category | MS | CS | MC | Se | Mn | Ca |
|---|---|---|---|---|---|---|
| Healthy | — | — | — | — | — | — |
| Exposure of selenium deficiency | ↑ | ↑ | x | ↓ | x | x |
| Exposure of selenium toxicity | ↓ | ↓ | x | ↑ | x | x |
| Exposure of manganese deficiency | ↓ | x | ↓ | x | ↓ | x |
| Exposure of manganese toxicity | ↑ | x | ↑ | x | ↑ | x |
| Exposure of calcium deficiency | x | ↓ | ↑ | x | x | ↓ |

Primary risk indicators: category indicator in Mn, Se and Ca which has abnormal result, and indicator in MS, CS and MC which has abnormal result in categories, all of them are determined by mathematical functions and probabilities;

Secondary risk indicators: indicator in MS, CS, and MC which has abnormal result beyond the category;

Reference indicators: indicator in Mn, Se, and Ca which has abnormal result beyond the category. These indicators provide only reference information beyond the category.

Under the environmental exposure risk, the number of abnormal results in indicators varies depending on the complexity of an actual exposure situation. In the risk category, the number of the primary risk indicators is related to the risk exposure situation. As the exposure level is greater and the time is longer, there are more abnormal results in indicators. Therefore, in the category, there are one to three primary risk indicators after exposure. In addition, the environmental risk exposure assessment system uses mathematical function to assess the exposure risk level.

According to this, comprehensive assessment results for the assessed subjects include: \textcircled{1}. categorizing exposure risk; \textcircled{2}. determining an exposure risk level in the category; \textcircled{3}. determining primary risk indicators, secondary risk indicators in the category, and other reference indicators.

The exposure risk categories and indicators are summarized in Table 2:

TABLE 2
Primary risk categories and indicators of exposure
risks in complex Mn, Se, and Ca environment

| | | | | Secondary risk | Reference | |
|---|---|---|---|---|---|---|
| | | | | indicators | indicators | |
| Exposure risk category | Primary risk indicators | | | (Unknown) | (Unknown) | |
| Healthy | None | | | None | None | |
| Exposure risk of selenium deficiency | ↓Se | ↑MS | ↑CS | MC | Mn | Ca |
| Exposure risk of selenium toxicity | ↑Se | ↓MS | ↓CS | MC | Mn | Ca |
| Exposure risk of manganese deficiency | ↓Mn | ↓MS | ↓MC | CS | Se | Ca |
| Exposure risk of manganese toxicity | ↑Mn | ↑MS | ↑MC | CS | Se | Ca |
| Exposure risk of calcium deficiency | ↓Ca | ↓CS | ↑MC | MS | Mn | Se |

Environmental exposure risk level (Table 3): a risk mathematical function value of the assessed subject is between the critical value of the healthy crowd and the critical value of a disease, and as the risk mathematical function value deviates from the critical value of the healthy crowd farther, the exposure risk level is more severe; as the risk mathematical function value is more approximate to the critical value of a disease, the exposure risk level is more severe.

TABLE 3
Assessment of the exposure risk level in complex Mn, Se, and Ca environment

Environmental
exposure risk

| category determined | Level of environmental exposure risk | |
|---|---|---|
| by mathematical | Critical value of the | |
| function and | function | |
| probability | (Critical value of | Confidence interval |
| Healthy | the risk level) | (Single indicator range) |
| Exposure risk of | When the | When a category indicator test value is less than |
| selenium deficiency | assessment | (<) the lower limit of the healthy crowd or is |
| | function value | combined with another test value beyond the |
| | deviates from the | range, a single factor exposure risk is increased |
| | critical value of the | beyond the range. |
| Exposure risk of | healthy crowd, the | When a category indicator test value is greater |
| selenium toxicity | exposure risk level | than (>) the upper limit of the healthy crowd or is |
| | of the primary risk | combined with another test value beyond the |
| | is increased. | range, a single factor exposure risk is increased |
| | | beyond the range. |
| Exposure risk of | | When a category indicator test value is less than |
| manganese | | (<) the lower limit of the healthy crowd or is |
| deficiency | | combined with another test value beyond the |
| | | range, a single factor exposure risk is increased |
| | | beyond the range. |
| Exposure risk of | | When a category indicator test value is greater |
| manganese toxicity | | than (>) the upper limit of the healthy crowd or is |
| | | combined with another test value beyond the |
| | | range, a single factor exposure risk is increased |
| | | beyond the range. |
| Exposure risk of | | When a category indicator test value is less than |
| calcium deficiency | | (<) the lower limit of the healthy crowd or is |
| | | combined with another test value beyond the |
| | | range, a single factor exposure risk is increased |
| | | beyond the range. |

### 7. Operating Process of Comprehensive Assessment of an Environmental Exposure Risk for an Unknown Subject

The comprehensive assessment model and method may be served as the basis of a program to complete the entire calculation and reporting process by running the program, including: a main operating module of a instrument, an embedded operating module of an existing instrument, a main operating system of a reporting system, an embedded operating system of an existing reporting system, a main operating system of a online reporting and surveillance management system, an embedded operating system of an existing online reporting and surveillance management system, and the like. Testing data of each element can be obtained through a mass spectrometer, a spectrometer, or the like.

\textcircled{1}. For example, as a module of the instrument, a preset model is used to get final report, or the module of the instrument can be connected to the online reporting and surveillance management system or the reporting system to get final report.

A general process is as follows:
step 1: placing a specimen into a test vessel of an instrument loaded with an assessment module;
step 2: clicking an “Sample injection” button of the instrument, selecting a target element, and clicking an “Analysis” option;
step 3: transferring a test value to the assessment module and clicking an “Assessment analysis” option;
step 4: outputting an electronic test report, where the report result includes: \textcircled{1}. an exposure risk category; \textcircled{2}. an exposure risk level in the category; \textcircled{3}. primary risk indicators, secondary risk indicators in the category, and other reference indicators; and
step 5: outputting a printed test report.

\textcircled{2}. For example, as a module of the online reporting and surveillance management system, a general process is as follows:
step 1: obtaining test data through the instrument;
step 2: entering a online reporting and surveillance management system interface, filling in basic information of a tested subject and the test data;
step 3: clicking an “Assessment analysis” option;
step 4: outputting an electronic test report, where the report result includes: \textcircled{1}. an exposure risk category; \textcircled{2}. an exposure risk level in the category; \textcircled{3}. primary risk indicators, secondary risk indicators in the category, and other reference indicators; and

Step 5: filling in environmental exposure information of a primary risk category of the tested subject, selecting a preset condition option under the category, and confirming, and a printed test report can be output;

step 6: clicking a “Report” option, so that the tested subject information and result are transmitted to an administrator server via the network; and

step 7: administrator summarize and analyze the reported data to complete a management report, and regularly calibrating the instrument which using a preset model.

1. A non-invasive assessment model for exposure risk of manganese, selenium, and calcium in a complex environment, comprising:
a. sampled subject selection for the model, comprising selecting sampled subjects from a healthy crowd, a crowd in a selenium deficiency environment, a crowd in a selenium toxicity environment, a crowd in a manganese deficiency environment, a crowd in a manganese toxicity environment, and a crowd in a calcium deficiency environment;
b. data collection, comprising collecting, by an instrument, manganese content data, selenium content data, and calcium content data of each sampled subject from hair, toenails or fingernails of the sampled subject, wherein the instrument is a mass spectrometer or a spectrometer;
c. data processing in a processor, comprising:
i: dividing, by the processor executing a computer program, the collected manganese content data, selenium content data, and calcium content data of each sampled subject in element pairs to obtain modeling data comprising at least one ratio of manganese/selenium (MS), calcium/selenium (CS), and manganese/calcium (MC);
ii, wherein processing in the obtained ratios between any two elements of the manganese, selenium, and calcium content data is consistent across sampled subjects;
d. establishing an exposure risk assessment model and a risk level assessment model, comprising: establishing, by the processor executing the computer program, a group of empirically derived discriminant equations to serve as the exposure risk assessment model and a linear regression equation to serve as the risk level assessment model, based on modeling data obtained from pre-classified subject populations representing the selected environmental exposure categories, the discriminant equations being structured to classify subjects based on the individual subject's risk profile, the linear regression equation being structured to determine a level indicating the subject's proximity to a disease status, based on the subject's risk profile;
e. risk assessment in the processor, comprising:
i: collecting manganese content data, selenium content data, and calcium content data from hair, toenails, or fingernails of a subject being assessed, using the mass spectrometer or the spectrometer;
ii. transmitting the collected data to the processor executing the computer program, either manually or directly, and processing the data to generate assessment indicators consistent with the modeling data;
iii. substituting the assessment indicators into the discriminant equations to classify the subject into a primary exposure risk category, the category being selected from:
healthy, an exposure risk of selenium deficiency in environment, an exposure risk of selenium toxicity in environment, an exposure risk of manganese deficiency in environment, an exposure risk of manganese toxicity in environment, and an exposure risk of calcium deficiency in environment; and
iv. outputting, by the processor executing the computer program, an assessment report comprising the classified primary exposure risk category and corresponding exposure risk level, the report being displayed on a display device or printed;
f. determining the level indicating the subject's proximity to the disease status, comprising: substituting, by the processor executing the computer program, the assessment indicators into the linear regression equation to determine a risk mathematical function value, and determining the level of the subject's proximity to the disease status under the primary exposure risk category based on the risk mathematical function value and a critical value of a disease, wherein the closer the risk mathematical function value is to the critical disease value, the more severe the subject's proximity to the disease status,
wherein the processor executing the computer program comprises a trained modeling engine configured to receive biological input from the mass spectrometer or the spectrometer and to output the primary exposure risk category of a categorized environmental exposure risk and the level which the subject is exposure to disease status under the primary exposure risk category for the subject under assessment.

2. (canceled)

3. The non-invasive assessment model in the processor for exposure risk of manganese, selenium, and calcium in a complex environment according to claim 1, wherein during data processing, the element content data collected from each sampled subject is recorded in scientific notation.

4. (canceled)

5. The non-invasive assessment model in the processor for exposure risk of manganese, selenium, and calcium in a complex environment according to claim 3, wherein the calcium content data collected from each sampled subject is recorded in scientific notation with 100.

6-10. (canceled)

11. The non-invasive assessment model in the processor for exposure risk of manganese, selenium, and calcium in a complex environment according to claim 1, wherein a result of the group of discriminant equation is used to determine the primary risk category through a posterior probability.

12. (canceled)

13. The non-invasive assessment model in the processor for exposure risk of manganese, selenium, and calcium in a complex environment according to claim 1, wherein an abnormal result of indicator is determined according to confidence interval of single element and confidence interval of a ratio between elements in pairs, to determine a secondary risk.

14-21. (canceled)

22. The non-invasive assessment model in the processor for exposure risk of manganese, selenium, and calcium in a complex environment according to claim 1, wherein the exposure risk assessment model comprises:
$$\{ \begin{matrix}{Y}_{1} = {C}_{01} + {C}_{11} {X}_{1} + {C}_{21} {X}_{2} + {C}_{31} {X}_{3} \\ {Y}_{2} = {C}_{02} + {C}_{12} {X}_{1} + {C}_{22} {X}_{2} + {C}_{32} {X}_{3} \\ {Y}_{3} = {C}_{03} + {C}_{13} {X}_{1} + {C}_{23} {X}_{2} + {C}_{33} {X}_{3} \\ {Y}_{4} = {C}_{04} + {C}_{14} {X}_{1} + {C}_{24} {X}_{2} + {C}_{34} {X}_{3} \\ {Y}_{5} = {C}_{05} + {C}_{15} {X}_{1} + {C}_{25} {X}_{2} + {C}_{35} {X}_{3} \\ {Y}_{6} = {C}_{06} + {C}_{16} {X}_{1} + {C}_{26} {X}_{2} + {C}_{36} {X}_{3}\end{matrix}$$
where the Cⱼₖ (j=0, 1, 2, and 3; k=1, 2, 3, 4, 5, and 6) is discrimination coefficient obtained by the pooled covariance of the modeling data, the X₁, X₂ and X₃ is assessment data collected by the manganese content data, the selenium content data, and the calcium content data of the subject in assessing consist with the process of modeling data when the exposure risk assessment model is established,
wherein performing, by the processor executing the computer program, risk assessment on a subject being assessed to determine a primary risk category of the subject in assessing comprises:
the assessment data of the subject in assessing are substituted into the exposure risk assessment model to calculate a function value Yₖ, a category of the subject in assessing is classified into the largest mathematical function value, a posterior probability Pₖ of the k category is calculated, and the primary risk category of the subject in assessing is classified into a risk category corresponding to the largest posterior probability, wherein k=1 means that the subject in assessing is in the primary risk category of a healthy person, k=2 means that the subject in assessing is in the primary risk category of the exposure risk of selenium deficiency in environment, k=3 means that the subject in assessing is in the primary risk category of the exposure risk of selenium toxicity in environment, k=4 means that the subject in assessing is in the primary risk category of the exposure risk of manganese deficiency in environment, k=5 means that the subject in assessing is in the primary risk category of the exposure risk of manganese toxicity in environment, and k=6 means that the subject in assessing is in the primary risk category of the exposure risk of calcium deficiency in environment.

23. The non-invasive assessment model in the processor for exposure risk of manganese, selenium, and calcium in a complex environment according to claim 1, wherein a result of the group of discriminant equation is used to determine the primary risk category through a function value.

24. The non-invasive assessment model in the processor for exposure risk of manganese, selenium, and calcium in a complex environment according to claim 13, wherein the abnormal result of indicator being determined according to confidence interval of single element and confidence interval of the ratio between elements in pairs, to determine a secondary risk comprises:
establishing confidence interval of the indicators, comprising, calculating, by the processor executing the computer program, an upper limit and a lower limit of confidence interval of MS, CS, MC, S, M, and C of the healthy crowd respectively, based on the modeling data obtained from pre-classified subject populations representing the selected environmental exposure categories;
determining the secondary risk of the subject in assessing, comprising, comparing, by the processor executing the computer program, the collected data of the subject in assessing and the assessment indicators with the corresponding confidence intervals to determine the secondary risk, wherein when the collected data or assessment indicators falls outside the corresponding confidence intervals, the element associated with that data or indicator is taken as the secondary risk.

25. The non-invasive assessment model in the processor for exposure risk of manganese, selenium, and calcium in a complex environment according to claim 1, wherein the critical value of a disease comprises an upper limit of confirmed case in clinical diagnosis,
the determining the level which the subject in assessing is exposure to disease status under the primary exposure risk category based on the risk mathematical function value and the critical value of the disease comprises:
determining, where a risk critical value of dependent variable of an exposed group exceeds a risk critical value of dependent variable of a healthy group, whether the risk mathematical function value is greater than or equal to a distinguishing threshold value, and determining the subject in assessing being exposure to disease status if the risk mathematical function value is greater than or equal to the distinguishing threshold value; and
determining, when the subject in assessing being exposure to disease status, the level which the subject is exposure to disease status under the primary exposure risk category based on the risk mathematical function value and the upper limit of confirmed case in clinical diagnosis, wherein a larger risk mathematical function value indicates closer proximity to the upper limit associated with confirmed cases in clinical diagnosis, and corresponds to a greater level indicating the subject's proximity to the disease status.

26. The non-invasive assessment model in the processor for exposure risk of manganese, selenium, and calcium in a complex environment according to claim 25, wherein the distinguishing threshold value is a mean value of the risk critical value of dependent variable of the healthy group and the risk critical value of dependent variable of the exposed group.

27. The non-invasive assessment model in the processor for exposure risk of manganese, selenium, and calcium in a complex environment according to claim 25, wherein the risk critical value of dependent variable of the exposed group for one type of the crowd in a selenium deficiency environment, the crowd in a selenium toxicity environment, the crowd in a manganese deficiency environment, the crowd in a manganese toxicity environment is a value obtained by substituting the modeling data of the type into the linear regression equation, and the risk critical value of dependent variable of the healthy group is a value obtained by substituting the modeling data of the healthy crowd into the linear regression equation.

28. The non-invasive assessment model in the processor for exposure risk of manganese, selenium, and calcium in a complex environment according to claim 1, wherein the critical value of a disease comprises a lower limit of confirmed case in clinical diagnosis, the determining the level which the subject in assessing is exposure to disease status under the primary exposure risk category based on the risk mathematical function value and the critical value of the disease comprises:
determining, where a risk critical value of dependent variable of an exposed group is less than a risk critical value of dependent variable of a healthy group, whether the risk mathematical function value is less than or equal to a distinguishing threshold value, and determining the subject in assessing being exposure to disease status if the risk mathematical function value is less than or equal to the distinguishing threshold value; and
determining, when the subject in assessing being exposure to disease status, the level which the subject is exposure to disease status under the primary exposure risk category based on the risk mathematical function value and the lower limit of confirmed case in clinical diagnosis, wherein a smaller risk mathematical function value indicates closer proximity to the lower limit associated with confirmed cases in clinical diagnosis, and indicates that the subject is more proximate to the disease status.

29. The non-invasive assessment model in the processor for exposure risk of manganese, selenium, and calcium in a complex environment according to claim 28, wherein the distinguishing threshold value is a mean value of the risk critical value of dependent variable of the healthy group and the risk critical value of dependent variable of the exposed group.

30. The non-invasive assessment model in the processor for exposure risk of manganese, selenium, and calcium in a complex environment according to claim 28, wherein the risk critical value of dependent variable of the exposed group for one type of the crowd in a selenium deficiency environment, the crowd in a selenium toxicity environment, the crowd in a manganese deficiency environment, the crowd in a manganese toxicity environment is a value obtained by substituting the modeling data of the type into the linear regression equation, and the risk critical value of dependent variable of the healthy group is a value obtained by substituting the modeling data of the healthy crowd into the linear regression equation.
